(19) United States
(12) Patent Application Publication
(10) Pub. No.: US 2012/0039821 A1
(43) Pub. Date: Feb. 16, 2012

(54) Dental Activating Gel

(75) Inventors: Irwin Smigel, New York, NY (US); Lucia Smigel, New York, NY (US); Said Raziq, Wentzville, MO (US)

(73) Assignee: ROBELL RESEARCH, INC., New York, NY (US)

(21) Appl. No.: 13/209,855

(22) Filed: Aug. 15, 2011

Related U.S. Application Data
(60) Provisional application No. 61/373,433, filed on Aug. 13, 2010.

(51) Int. Cl. A61K 8/18; A61K 8/19; A61K 8/24; A61K 8/39; A61C 17/00; A61K 8/84; A61K 8/49; A61K 8/368; A61Q 11/00; A61K 8/22; A61K 8/90

(52) U.S. Cl. 424/53; 424/49; 424/57; 424/54; 424/55; 132/286

(57) ABSTRACT

A liquid composition that is applied to teeth in advance of a whitening gel or paste. The liquid composition is preferably in a viscous gel form, such that when the composition is applied to teeth, it does not flow quickly off the teeth. The composition contains ne diluent; oxidizer; buffering agent; suspending agent; emollient; surface active agent; germicide; gelling agent; optionally one or more flavoring components selected from flavor stabilizers, sweeteners, and taste enhancers; and pH control agent.

### PRIORITY

This application claims the benefit of U.S. Provisional Application Ser. No. 61/373,433, filed Aug. 13, 2010.

### FIELD OF INVENTION

This invention is an activating gel for use in dental whitening and cleansing applications and a delivery system to contain and apply the activating gel.

### BACKGROUND

Dental whitening products often consist of gels and pastes, either applied by dentists or specialists in an office setting or applied by individuals at home. The most common dental whitening product is toothpaste, applied by a toothbrush. Toothpaste products, whether or not they provide whitening, often include cleaning and cavity protection components, such as fluoride. Toothbrush bristles remove food particles and dental plaque from teeth, and apply the toothpaste to the teeth at the same time.

Certain substances found on teeth interfere with dental whitening products or prevent the dental whitening products from contacting the tooth enamel. Such substances are preferably removed prior to use of conventional dental whitening products. One such substance is layer of protein called a pellicle. Dental pellicle or protein pellicle forms on the enamel of teeth by the binding of certain salivary glycoproteins. Dental pellicle protects teeth from certain acids produced in the mouth after consuming carbohydrates. However, dental pellicle also provides an easier means for bacteria to attach to teeth, including bacteria which causes dental caries and odors.

A dental product which removes the dental pellicle can provide more direct contact with tooth enamel, thereby providing improved whitening action directly on the tooth enamel, without interference from the dental pellicle.

Application of dental whitening products can be in a variety of methods. Typical home use applications include the use of toothbrushes, trays and strips. As maximum coating of a tooth is desired, trays provide uniform coverage of the tooth. However, the use of a tray may be uncomfortable. Strips with whitening agents do not provide uniform coverage and may be dislodged before the preferred time is elapsed.

Dental whitening products that are applied by toothbrush have the disadvantage that the brush bristles each provide a small area of contact with the tooth enamel. However, the brush bristle is important for the function of removing particles that may have become attached to the tooth enamel, between teeth, or between teeth and gums.

It is desired to have a dental product that can help remove dental pellicle prior to application of toothpaste or other whitening products.

It is desired to have a dental product that can prepare teeth for later stages of whitening.

It is desired to have a soft applicator for dental products with a wide surface to cover a greater area of teeth.

It is desired to have a portable applicator for dental products that contains the dental product in a single-use applicator.

### SUMMARY OF THE INVENTION

The present invention is a liquid composition that is applied to teeth in advance of a whitening gel or paste. The liquid composition is preferably in a viscous gel form, such that when the composition is applied to teeth, it does not flow quickly off the teeth, preferably the viscosity is in the range of A-2 to B on the Gardner Viscosity Bubble Scale, which corresponds to about 22 to 65 cps. One method of applying the composition is through a soft swab, preferably permeable to the composition. In one delivery system the composition is composed of two pieces, one which retains the composition in a sealed chamber until the composition is ready for use, and the other which is composed of a soft permeable swab, mounted on a hollow shaft. The sealed chamber has a pre-determined break point, allowing one end to be removed, exposing the chamber containing the composition. The hollow shaft is inserted into the chamber and as the shaft is inserted, the periphery of the shaft displaces the composition, forcing the composition into the interior of the hollow shaft. The composition travels up the interior of the hollow shaft and permeates the soft swab, resulting in the composition on the external surface of the swab, ready for application to the teeth.

### DETAILED DESCRIPTION OF THE PREFERRED EMBODIMENT

The invention preferably consists of a viscous liquid composition of a diluent, an oxidizer, a buffering agent, one or more emollients, a gelling agent, one or more suspending agents, a germicide, a surface active agent, and one or more flavoring agents. The diluent is water, preferably deionized. The oxidizer is preferably hydrogen peroxide, although urea hydrogen peroxide (aka urea carbamide) may be used as a suitable alternative. The buffering agent may be selected from citrate and carbonate buffers, preferably (a) containing (or formed in situ) sodium citrate or potassium citrate or both (b) containing (or formed in situ sodium bicarbonate or potassium bicarbonate or both; most preferably containing (or formed in situ) sodium bicarbonate. The emollients are one or more of glycerine, sorbitol, polyethylene glycol, propylene glycol, and dipropylene glycol. The gelling agent is preferably carbopol 934p or carbopol 980, most preferably carbopol 934p The suspending agent is one or more of tetra potassium pyrophosphate, potassium carbonate and sodium tripolyphosphate. The germicide is water soluble and readily rinsible quaternary ammonium germicidal compound such as cetyl pyridinium chloride or benzalkonium chloride orthe like, most preferably cetyl pyridinium. The surface active agents are one or more of a poloxamer (preferably poloxamer 407) and a polysorbate (tween 20, also known as polysorbate 20) as long as they are water soluble and readily rinsable. The flavoring agents are one or more of sweetener, a flavor stabilizer, and flavors for the composition. The sweetener is preferably sodium sacchrinate. The flavor stabilizer is preferably sodium benzoate. The composition may further include an agent for pH control, such as a food acceptable acid, preferably a food acceptable organic acid, most preferably malic acid.

In the best mode known to the inventors, the composition ingredients are in the percentages set forth in Table 1.

TABLE 1

| | Approximate | | Specific highly |
|---|---|---|---|
| | percentage in | Preferred % | preferred |
| Ingredient | composition | ranges | formulation % |
| Deionized Water | 40%-80% | 40-50%; | 77.38% |
| | | 76.60%-78.15% | |
| Hydrogen Peroxide | 0.1%-4% | 0.1%-0.4% | 0.80% |
| | | 0.76%-0.84% | |
| Sodium Bicarbonate | 0.1%-0.3% | 0.142%-0.158% | 0.15% |
| Tetra Potassium | 0.01%-0.5% | 0.047%-0.053% | 0.05% |
| Pyrophosphate | | | |
| Potassium Carbonate | 0.01%-0.5% | 0.047%-0.053% | 0.05% |
| Sodium Benzoate | 0.01%-0.2% | 0.095%-0.105% | 0.10% |
| Sodium | 0.01%-0.3% | 0.047%-0.053% | 0.05% |
| Tripolyphosphate | | | |
| Poloxamer 407 | 0.1%-1.0% | 0.190%-0.210% | 0.20% |
| Cetyl Pyridinium | 0.01%-0.2% | 0.066-0.074 | 0.07% |
| Chloride | | | |
| Sodium Sacchrinate | 0.1%-0.5% | 0.24%-0.26% | 0.25% |
| Carbopol 934p | 0.04%-0.6% | 0.048?-0.52 | 0.5% |
| Tween 20 | 0.5%-2.0% | 0.095%-0.105% | 0.1% |
| Glycerine | 5%-20% | 9.50%-10.50% | 10.0% |
| Sorbitol | 5%-15% | 9.50%-10.50% | 10.0% |
| Flavor | 0.1%-0.3% | 0.095%-0.105% | 0.1% |
| Malic Acid | 0.1%-0.5% | 0.190%-0.21% | 0.2% |

Upon application, the composition acts as an exfoliator for teeth, removing surface stains and the dental pellicle. The composition prepares the teeth for the subsequent application of whitening products such as toothpastes and whitening gels.

One delivery system for the composition is composed of a sliding plunger partially contained in a retaining tube with the composition stored inside the retaining tube. An applicator, which may be a swab or sponge like material having a hollow tube communicator with the internal portion of the retaining tube, is attached or can be attached to the retaining tube, generally after the device is opened. On depression of the plunger into the retaining tube, the composition is forced to migrate through the device into the interior of swab or sponge like material from which it can migrate to the surface of the swab or sponge like material for application to the tooth surface. Such plunger type systems are generally known in the art and available from a number of manufacturers.

Other methods of applying or storing and applying the composition as will be apparent to those of ordinary skill in the art may of course be used. Such alternate methods include, but are not limited to, supplying the composition in a compressible tube (analogous to a toothpaste tube) or in a dispenser container, which may either be pressurized or unpressurized, or a pour bottle or container where the user dispenses an amount of the composition into an open container or onto an application device. The user, if dispensing the composition into an open container, then uses an application device to transfer a portion of the composition from the open container to tooth surfaces, or if dispensing the composition directly onto an application device then uses the application device to transfer the composition to tooth surfaces. Typical application devices are, without limitation, devices such as toothbrushes, swabs, mouth trays, etc. Still further alternate application methods include the use of towelettes or cloths of appropriate fabric that are impregnated with the composition of the present invention. When such impregnated towelettes are used, the towelette is sealed within an impermeable wrapper of the type known in the art. The user unwraps the towelette and rubs it onto the tooth surface to be treated.

### EXAMPLES

### Example 1

### Preparation of a Preferred Formulation of the Present Invention

In this Example, the following formulation is prepared.

| Ingredient | % |
|---|---|
| Deionized Water | 77.38% |
| Hydrogen Peroxide | 0.80% |
| Sodium Bicarbonate | 0.15% |
| Tetra Potassium Pyrophosphate | 0.05% |
| Potassium Carbonate | 0.05% |
| Sodium Benzoate | 0.10% |
| Sodium Tripolyphosphate | 0.05% |
| Poloxamer 407 | 0.20% |
| Cetyl Pyridinium Chloride | 0.07% |
| Sodium Sacchrinate | 0.25% |
| Carbopol 934p | 0.5% |
| Tween 20 | 0.1% |
| Glycerine | 10.0% |
| Sorbitol | 10.0% |
| Flavor | 0.1% |
| Malic Acid | 0.2% |

The deionized water is charged into a stainless steel vessel. After turning on an agitator, add the sodium bicarbonate, the tetra potassium pyrophosphate and potassium carbonate and continue agitation until all solids have dissolved and formed a true solution. Then add the sodium benzoate, the sodium tripolyphosphate and continue agitation until all solids have dissolved forming a true solution. The glycerine and sorbitol are then added and agitation is continued until a homogeneous liquid is obtained. Then add the poloxamer 407, the cetyl pyridinium chloride, the sodium saccharinate and the tween 20 components and continue agitation until a homogenous product is obtained. Agitation is then stopped and the hydrogen peroxide is added. Once the hydrogen peroxide is added, restart the agitation and continued until the liquid is uniform and clear in appearance. The carbopol 934p is then sprinkled onto the liquid in the vessel and mixed until all of the carbopol component has been hydrated to a clear viscous gel free of any suspended matter. The malic acid component is then added with agitation until it is completely in solution. The flavor components are then added and mixed until the flavor is in solution. The resulting composition is in the form of a gel with a pH of about 4.5 to 6.0. If desired, the flavoring components may be omitted.

While certain novel features of the present invention have been shown and described, it will be understood that various omissions, substitutions and changes in the forms and details of the device illustrated and in its operation can be made by those skilled in the art without departing from the spirit of the invention.

I claim:

1. A dental composition comprising:
a) at least one diluent;
b) at least one oxidizer;
c) at least one buffering agent;
d) at least one suspending agent;
e) at least one emollient;
f) at least one surface active agent;
g) at least one germicide;
h) at least one gelling agent;
i) optionally one or more flavoring components selected from flavor stabilizers, sweeteners, and taste enhancers; and
j) a pH control agent.

2. The composition of claim 1, where the diluent is deionized water.

3. The composition of claim 1, where the oxidizer is hydrogen peroxide.

4. The composition of claim 1, where the buffering agent is sodium bicarbonate.

5. The composition of claim 1, where the suspending agent is one or more of tetra potassium pyrophosphate, potassium carbonate, and sodium tripolyphosphate.

6. The composition of claim 1, where the emollient is one or more of glycerine, sorbitol, polyethylene glycol, propylene glycol, and dipropylene glycol.

7. The composition of claim 1, where the surface active agent is one or more of poloxamer 407 and tween 20.

8. The composition of claim 1, where the germicide is cetyl pyridinium chloride.

9. The composition of claim 1, where the gelling agent is carbopol 934p.

10. The composition of claim 1, where the flavor stabilizer is sodium benzoate.

11. The composition of claim 1, where the sweetener is sodium saccharinate.

12. The composition of claim 1, where the taste enhancer is flavoring.

13. The composition of claim 1, where the pH control agent is malic acid.

14. The composition of claim 1 comprising:

| | |
|---|---|
| a) Deionized Water | 77.38% |
| b) Hydrogen Peroxide | 0.80% |
| c) Sodium Bicarbonate | 0.15% |
| d) Tetra Potassium Pyrophosphate | 0.05% |
| e) Potassium Carbonate | 0.05% |
| f) Sodium Benzoate | 0.10% |
| g) Sodium Tripolyphosphate | 0.05% |
| h) Poloxamer 407 | 0.20% |
| i) Cetyl Pyridinium Chloride | 0.07% |
| j) Sodium Sacchrinate | 0.25% |
| k) Carbopol 934p | 0.5% |
| l) Tween 20 | 0.1% |
| m) Glycerine | 10.0% |
| n) Sorbitol | 10.0% |
| o) Flavor | 0.1% |
| p) Malic Acid | 0.2%. |

15. A method of cleaning or whitening teeth which comprises applying to said teeth a composition of claim 1.

16. The method of claim 15 wherein the composition of claim 1 is a pre-treatment for the subsequent use of another tooth whitening or cleaning product.

17. A method of removing, at least in part, tooth stain or pellicle or both from a tooth surface comprising applying to said tooth surface a composition of claim 1.

18. An application kit for the application of the composition of claim 1 comprising a dispenser for dispensing the composition of claim 1, the composition of claim 1 contained therein, and an applicator component for the application of the composition of claim 1 to a tooth surface in need of having the composition of claim 1 applied thereto.
